(19) United States
(12) Patent Application Publication
(10) Pub. No.: US 2022/0062357 A1
(43) Pub. Date: Mar. 3, 2022

(54) METHOD AND COMPOSITION AGAINST VIRUS INFECTIONS WITH ACTIVATED INNATE LYMPHOID CELLS (ILCS)

(71) Applicants: Purdue Research Foundation, West Lafayetter, IN (US); The United States of America, as represented by the Secretary, Department of Health and Human Servic, Rockville, MD (US)

(72) Inventors: Mittal Suresh, West Lafayette, IN (US); Suryaprakash Sambhara, Atlanta, GA (US); Weiping Cao, Atlanta, GA (US); Wadzanai Mboko, Atlanta, GA (US); Ekramy Sayedahmed, West Lafayette, IN (US)

(73) Assignees: Purdue Research Foundation, West Lafayetter, IN (US); The United States of America, as represented by the Secretary, Department of Health and Human Servic, Rockville, MD (US)

(21) Appl. No.: 17/409,962

(22) Filed: Aug. 24, 2021

Related U.S. Application Data
(60) Provisional application No. 63/070,856, filed on Aug. 27, 2020.

(51) Int. Cl. A61K 35/761; A61P 31/12; A61P 31/16

(52) U.S. Cl. CPC A61K 35/761; A61P 31/16; A61P 31/12

(57) ABSTRACT

The present disclosure provides method and composition for protection, prevention, and/or treatment against viral infections via activation of ILCs induced by certain virus, including but not limited to, influenza and/or non-replicating adenoviruses. The present disclosure further provides that activation of ILCs is an intervention strategy against not only influenza viral infectious epidemic and/or pandemic before a strain-matched vaccine is available but also against other viruses for which prophylactic vaccines may not be available.

### CROSS-REFERENCE TO RELATED APPLICATIONS

This application claims the benefit of U.S. Provisional Application No. 63/070,856, filed Aug. 27, 2020, which is incorporated herein by reference in its entirety.

### GOVERNMENT SUPPORT CLAUSE

This invention was made with government support under grant AI059374 awarded by the National Institutes of Health. The government has certain rights in the invention.

### FIELD OF INVENTION

The present disclosure relates generally to innate lymphoid cells (ILCs) as potent effector cells, and a method of use thereof, for preventing, treatment, and/or protection against viral infections.

### BACKGROUND OF THE INVENTION

Innate lymphoid cells (ILCs) rose to prominence as potent regulators of innate immunity in the respiratory mucosa, gastrointestinal tract as well as the skin^{1,2}. ILCs exert their action via cytokine production and cytolytic function and can have profound effects on the local immune response³. Recently, key roles for ILCs have been described in inflammation and tumor immunology^{4,5}, and emerging work has revealed potential importance in viral infections. Three classes of ILCs have been described which are characterized by transcription factor expression as well as cytokine production upon activation⁶. A high level of plasticity of ILCs has also been reported, which can be attributed to the cytokine milieu^{1,7}. Earlier studies indicated a predominant role for ILC2 cells in tissue repair, pulmonary hyper-responsiveness, airway epithelial integrity, and tissue homeostasis during influenza infection in mice^{8,9}, and a potential role for ILC1 activation and regulation in influenza¹⁰. Very limited information is available on the role of ILC3. These studies represent initial efforts to understand ILC biology in the context of influenza infection; however, the protective role of ILCs in influenza infection remains unaddressed.

Influenza is the cause of considerable morbidity and mortality globally, resulting in an estimated 291,243 to 645,832 fatalities annually and is a pathogen of significant public health importance. Certain groups, i.e., infants and pregnant young women, older adults and immunocompromised, are especially at risk for severe disease¹². Vaccination remains the most effective measure against influenza infection¹³. Despite immunization being the most effective and economic prophylactic approach, vaccines often provide less than optimal defense against influenza, with efficacies ranging from 10-60% as the emergence of a new strain could leave even previously immunized individuals at risk, as was observed with H3N2 variant strains in 2012¹⁴.

In addition to annual epidemics, the occurrence of pandemics is also a major concern as demonstrated by three major influenza pandemics in the 20^{th} century in 1918, 1957 and 1968, and the first influenza pandemic of the 21^{st} century in 2009 that spread worldwide in a short period causing significant morbidity and mortality. Although a pandemic was declared by WHO on Jun. 12, 2009, the strain-matched vaccine became available only during October of 2009, which was too late for public health intervention strategies to prevent the spread of the pandemic virus by vaccination. Besides, circulation and infection of humans with novel avian influenza viruses from subtypes H5N1, H7N7, H7N1, H7N3, H7N9, and H9N2 with significant case fatalities (ranges from 52% for H5N1 and 38% for H7N9) is a cause for concern as global population lacks immunity to these viruses.

Hence, antiviral drugs, a neuraminidase inhibitor, oseltamivir (Tamiflu®) and newly introduced viral polymerase inhibitor, baloxavir marboxil (Xofluza) take center stage in public health intervention strategies; however, oseltamivir- and baloxavir-resistant influenza seasonal viruses have been reported, and a pandemic with a drug-resistant virus will result in significant case fatalities¹⁵⁻²¹. Novel approaches to reduce disease burden and potential mortality associated with epidemic and pandemic influenza viruses are urgently needed.

### SUMMARY OF THE INVENTION

The present disclosure provides a method for protection, prevention, and/or treatment against viral infections with activated Innate Lymphoid Cells (ILCs) induced by certain viruses. In certain embodiments, such virus is non-replicating adenoviruses, such as HAd-ΔE1E3 or HAd-H7HA viruses, and/or influenza viruses, such as H1N1, H3N2, and/or H7N9 viruses.

Innate Lymphoid Cells (ILC) are potent effector cells that secrete chemokines and cytokines which facilitate the accumulation of a diverse repertoire of dendritic cells, macrophages, and neutrophils in the lungs to aid in innate immunity and the initiation of adaptive immune responses. The present disclosure provides that prior activation of ILCs can confer protection, prevention, and/or treatment against avian and seasonal influenza viral infections, as well as against other viruses (e.g., SARS-CoV2) for which prophylactic vaccines may not be available.

In certain embodiments, the present disclosure provides that Influenza virus or HAd-ΔE1E3 induces a robust activation of ILCs, and replication-defective adenovirus elicits a protective response against intranasal challenge with H7N9 and H1N1 influenza viruses. Such protective response is conferred in the absence of functional antibodies against the influenza virus or cross-reactive CD8 T cells against NP, a major CTL target.

In certain embodiments, the present disclosure provides that the influenza virus, such as H1N1 and H3N2 influenza viruses, induce activation of NK cells, ILC1, and ILC2 cells. In other embodiments, the present disclosure provides that the non-replicating adenovirus, such as HAd-ΔE1E3 or HAd-H7HA virus, induces diverse and robust activation of ILCs in lungs lasting for a period of time, for instance, about 7- to 28-days. The activated ILCs are human ILCs including ILC1, ILC2 and ILC3. It also shows that non-replicating adenovirus induces infiltration of immune cells or expression of inflammatory cytokine and antiviral genes in lungs.

The present disclosure further provides a method for protection, prevention, and/or treatment against viral infectious epidemic or pandemic challenges where a strain-matched vaccine is not available. The protection, prevention and/or treatment method comprises administering a population with a non-replicating adenovirus to induce diverse and robust activation of ILCs. In certain embodiments, the viral infectious epidemic or pandemic is caused by influenza viruses or any other viruses and/or variants, known or later developed. In certain embodiments, the non-replicating adenovirus is HAd-ΔE1E3 or HAd-H7HA virus. The protection, prevention and/or treatment method disclosed herein can contain the spread and reduce disease burden, limit disease severity, mitigate influenza-related deaths, reduce morbidity, or facilitate recovery.

A composition for protection, prevention and/or treatment against viral infection comprising an effective amount of a virus that is capable to induce diverse and robust activation of ILCs is also provided herein. In certain embodiment, the virus contained in the composition is non-replicating adenovirus, influenza virus, and/or other viruses and variants thereof, that can induce activation of one or more ILCs (e.g., NK cells, ILC1, ILC2 and ILC3) to induce infiltration of immune cells or expression of inflammatory cytokine and antiviral genes in lungs. In certain embodiments, the non-replicating adenovirus is HAd-ΔE1E3 or HAd-H7HA virus, and the influenza virus is H1N1 or H3N2 influenza viruses.

The composition disclosed herein can be in any suitable formula suitable for administration locally and/or systemically. In certain embodiments, the composition of the present disclosure can be administered via inhalation or injection. The composition of the present disclosure can be used for protection, prevention and/or treatment against viral infectious epidemic or pandemic challenges where a strain-matched vaccine is not available, to contain the spread and reduce disease burden, limit disease severity, mitigate viral-related deaths, reduce morbidity, or facilitate recovery.

### BRIEF DESCRIPTION OF THE DRAWINGS

FIGS. 1A-1D. Activation of human Innate Lymphoid Cells (ILCs) by Influenza virus or non-replicating adenovirus. Human PBMCs from 10 individuals were incubated with A/Taiwan/1/86 (H1N1), or A/HK/68 (H3N2) at an MOI of 3 PFU per cell for 16 hours (FIGS. 1A-1B), or infected with a non-replicating human adenovirus type C5 having E1 and E3 deletions (HAd-ΔE1E3) at an MOI of 10 PFU per cell for 16 hours (FIGS. 1C-1D). Different subsets of ILCs in PBMCs were identified using multi-parametric flow cytometry. The activation status of each ILC subset was measured by the surface expression of CD69 and CD25. The percentage of CD25⁺ or CD69⁺ ILCs were presented. *p≤0.05 **p≤0.01 ***p≤0.001.

FIGS. 2A-2G. Influenza virus or non-replicating adenovirus activates innate lymphoid cells (ILCs) in mice. BALB/c mice (5 animals per group) were administered with 733 PFU of A/HK/68 (H3N2) virus (FIGS. 2B-2D) or 5×10⁷ PFU of non-replicating adenovirus [HAd-ΔE1E3] (FIGS. 2E-2G) or PBS via the intranasal (i.n.) route, and the lungs were harvested at 3 and 7 days post-primary administration or 28 days after the booster administration and mononuclear cells were enriched by density centrifugation using Lymphoprep™. FIG. 2A. The gating strategy was used to identify ILC subsets. Cells were gated out of live, lineage⁻ CD45⁺lymphocytes. Cells were stained and analyzed by flow cytometry. As compared to PBS group, fold changes in (FIGS. 2B and 2E) NK cells [group 1 ILCs], (FIGS. 2C and 2F) group 2 ILCs, and (FIGS. 2D and 2G) group 3 ILCs were presented. Data are representative of two independent experiments. *p≤0.05 **p≤0.01 ***p≤0.001.

FIGS. 3A-3H. Non-replicating adenovirus induces infiltration of immune cells in the lungs. Mice (5 animal per group) were inoculated with 5×10⁷ PFU of non-replicating adenovirus (HAd-ΔE1E3) or PBS via the i.n route and lungs were harvested at 1, 3, 7, 14 and 28 days post primary inoculation, and 28 days post-boost. Lungs were analyzed for infiltration of immune cells by flow cytometry. As compared to PBS group, fold changes of the number of dendritic cells [DCs] (FIG. 3A) CD11 b⁺ DCs (FIG. 3B), CD103⁺ DCs (FIG. 3C), Plasmacytoid DCs (FIG. 3D), monocyte-derived DCs (FIG. 3E), neutrophils (FIG. 3F), alveolar macrophages (FIG. 3G) and interstitial macrophages (FIG. 3H) were presented. Fold changes were determined by comparing an absolute number of cells compared to PBS-treated group mice for each time point. Data are representative of two independent experiments. *p≤0.05 **p≤0.01 ***p≤0.001.

FIGS. 4A-4D. Non-replicating adenovirus induces expression of inflammatory cytokine and antiviral genes in the lungs. Mice were treated with 5×10⁷ PFU of non-replicating adenovirus (HAd-ΔE1E3) or PBS, and at indicated time post-treatment, expression levels of cytokines and known antiviral genes in the lung were examined by a qRT-PCR array. Three mice per group per time point were used. 10-fold upregulation compared to PBS used as a cutoff. Data are representative of two independent experiments. *p≤0.05 **p≤0.01 ***p≤0.001 ****p≤0.0001.

FIGS. 5A-5F. Non-replicating adenovirus protects against lethal challenge with H7N9 or H1N1 influenza virus in mice. BALB/c mice were treated with a non-replicating human adenoviral type C5 virus expressing hemagglutinin (HA) from Influenza virus A/Anhui/1/2013 (HAd-H7HA), non-replicating control adenovirus (HAd-ΔE1E3) or PBS via the intramuscular (i.m.) route (FIGS. 5A and 5B), or via the intranasal (i.n.) route (FIGS. 5C and 5D). Mice were boosted four weeks post-primary treatment and challenged four weeks after the boost with a lethal dose (5LD₅₀) of A/Anhui/1/2013 (H7N9) (FIGS. 5A-5D) or 2LD₅₀ of PR8 (H1N1) (FIGS. 5E-5F). (FIGS. 5A, 5C & 5E) Morbidity and (FIGS. 5B, 5D & 5F) mortality after challenge. Data are represented from one of three independent experiments with H7N9 challenge or 2 independent experiments with H1N1 challenge showing similar results.

FIGS. 6A-6D. Non-replicating adenovirus does not induce hemagglutination inhibiting (HI), virus-neutralizing or virus-binding antibodies against H7N9 influenza virus. Serum samples from mice that were inoculated intranasally (i.n.) with HAd-H7HA, HAd-ΔE1E3 or PBS were tested for (FIG. 6A) HI antibodies by HI assay, (FIG. 6B) neutralizing antibodies against H7N9 by micro-neutralization assay, and (FIG. 6C) H7N9 virus-binding antibodies by ELISA. Serum samples from mice treated with HAd-H7HA, HAd-ΔE1E3 or PBS (via La route or i.m. route) were tested for (FIG. 6D) neutralizing antibodies to human adenovirus C5 (HAd-C5) by micro-neutralization assay. Fifteen mice per group for panel FIGS. 6A and 6B, five mice per group for panel FIGS. 6C and 6D.

FIGS. 7A-7D. Non-replicating adenovirus induces activated CD4⁺ and CD8⁺ T cells, but not influenza NP-specific CD8⁺ T cells. Mice (5 animals per group) were inoculated with 5×10⁷ PFU of non-replicating adenovirus HAd-ΔE1E3 or PBS via the intranasal (i.n.) route, and the lungs were harvested, and single cell suspensions were stained for analyses by flow cytometry. The percentage of CD4⁺CD44⁺ T cells (FIG. 7A) and CD8⁺CD44⁺ T cells (FIG. 7B) were quantified by flow cytometry. FIG. 7C. Mice were treated with 5×10⁷ PFU of non-replicating adenovirus or infected with 15MID₅₀ of PR8. Nine days post-treatment, the lungs were harvested for assessing NP-specific CD8⁺ T cells by flow cytometry using NP-pentamers. FIG. 7D. Four weeks after the boost, mice were challenged with 20MID₅₀ of PR8, and NP-specific CD8⁺ T cells were measured at indicated time points post-challenge by flow cytometry. *P≤0.05 **P≤0.01 ***P≤0.001 ****P≤0.0001.

FIGS. 8A-8D. Inflammatory cytokine gene expression in the lungs of mice inoculated with non-replicating adenovirus. Mice were treated with 5×10⁷ PFU of non-replicating adenovirus (HAd-ΔE1E3) and at 1 day, 7 days, and 28 days post-treatment, and 28 days after the boost, the lungs were collected, and the expression levels of antiviral genes were examined by qRT-PCR array. FIGS. 8A-8D. Genes that were upregulated in treated animals on different time points post inoculation are presented. Two independent experiments were performed with similar results. Three mice per group per time point were used.

FIGS. 9A-9D. Antiviral gene expression in the lungs of mice inoculated with non-replicating adenovirus. Mice were inoculated with 5×10⁷ PFU of non-replicating adenovirus (HAd-ΔE1E3) and at 1 day, 7 days, and 28 days post-treatment, and 28 days after the boost, the lungs were collected, and the expression levels of antiviral genes were examined by qRT-PCR array. FIGS. 9A-9D. Genes that were upregulated in treated animals on different time points post-inoculation were presented. Two independent experiments were performed with similar results. Three mice per group per time point were used.

FIG. 10. Non-replicating adenovirus persistence in the lungs of inoculated mice. Mice were treated with 5×10⁷ PFU of non-replicating adenovirus (HAd-ΔE1E3), and the lungs were harvested at 4 weeks after the boost. DNA samples were prepared from lung homogenates, and adenovirus genomes in 50 ng of DNA were quantified by qPCR array using a set of HAd hexon primers. Data were combined from two independent experiments.

### DETAILED DESCRIPTION OF THE INVENTION

The present disclosure provides method and composition for protection, prevention, and/or treatment against viral infections with activated Innate Lymphoid Cells (ILCs) induced by certain viruses. In certain embodiments, such virus is non-replicating adenoviruses, such as HAd-ΔE1E3 or HAd-H7HA viruse, and/or influenza viruses, such as H1N1, H3N2, and/or H7N9 viruses. The present disclosure further provides a method and composition for protection, prevention, and/or treatment against viral infectious epidemic or pandemic challenges where a strain-matched vaccine is not available, by administering a population with a non-replicating adenovirus to induce diverse and robust activation of ILCs. In certain embodiments, the viral infectious epidemic or pandemic is caused by influenza viruses or any other viruses and/or variants, known or later developed. In certain embodiments, the non-replicating adenovirus is HAd-ΔE1E3 or HAd-H7HA virus. The protection, prevention and/or treatment method and composition disclosed herein can contain the spread and reduce disease burden, limit disease severity, mitigate influenza-related deaths, reduce morbidity, or facilitate recovery.

The following description of the embodiments is merely exemplary in nature and is in no way intended to limit the present disclosure, its application, or uses. Although specific terms are employed herein, they are used in a generic and descriptive sense only and not for purposes of limitation.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing certain aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the term “virus” refers to an infective agent that typically consists of a nucleic acid molecule in a protein coat and is able to multiply only within the living cells of a host. Most viruses have either DNA (Classes I and II) or RNA (Classes III-VI) as their genetic material. The nucleic acid may be single- or double-stranded. Examples of DNA viruses include but are not limited to: Adenoviruses, which cause infections in the upper respiratory tract and gastrointestinal tract in many animals; SV40 (simian virus 40), a monkey virus that was accidentally discovered in kidney cell cultures from wild monkeys used in the production of poliovirus vaccines; Herpesviruses, which cause various inflammatory skin diseases (e.g., chickenpox) and latent infections that recur after long intervals (e.g., cold sores and shingles); Human papillomaviruses (HPVs), which cause warts and other insignificant skin lesions and occasionally cause malignant transformation of cervical cells; and parvoviruses (from Latin parvo, “poor”). All the animal viruses belonging to classes III-VI have RNA genomes. RNA viruses include retroviruses, such as HIV (human immunodeficiency virus), and coronaviruses, such as SARS-CoV2 that causes COVID-19. RNA viruses, particularly retroviruses, are prone to mutate, meaning the set of genetic instructions that contain all the information that the virus needs to function can change as the virus spreads. As used herein, the term “virus” refers to all kind of viruses, now known or later discovered.

The most common viral infections are respiratory infections of the nose, throat, upper airways, and lungs caused by influenza, pneumonia and coronaviruses. As used herein, the term “non-replicating and/or replication-defective adenoviruses” refers to all adenoviruses, now known and classified or later discovered and/or identified, that gain an attenuated state wherein they can still be able to trigger the desired human immune responses but cannot replicate hi human cells.

There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. A pandemic can occur when a new and very different influenza A virus emerges that both infects people and has the ability to spread efficiently between people. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include A(H1N1) and A(H3N2). Influenza A subtypes can be further broken down into different genetic “clades” and “sub-clades.” As used herein, the term “influenza viruses” refer to all types, subtypes, glades and sub-clades of influenza viruses, now known, classified or later discovered and identified.

As used herein, the term “activated ILCs” refer to the numbers and activities of these ILCs are increased and/or upregulated in response to a host immunity by interacting with other immune cells and/or facilitating their interaction in multiple effectors.

As used herein, the term “effective amount” refers to an amount that is sufficient to achieve the desired modification of a physical property of the composition or material and/or achieving the desired level of reduction of withdrawal symptoms. The specific level in terms of wt % in a composition required as an effective amount will depend upon a variety of factors including the amount and type of formulation materials used in the disclosed compositions, amount and type of pharmaceutically acceptable excipients, and disorder being treated using the disclosed compositions.

As used herein, “protection/protecting,” “prevention/preventing,” and/or “treatment/treating” is an approach for obtaining beneficial or desired results including clinical results. For purposes of the present disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “protection/protecting,” “prevention/preventing” and/or “treatment/treating” is a reduction of pathological consequence of the disease. The methods provided herein contemplate any one or more of these aspects of protection, prevention, and/or treatment.

Methods for diagnosis and measurement of viral infections are well known in the art. In certain embodiments, the effective protection, preventing, and/or treatment is measure by the level of certain symptoms that is reduced by about 5% to about 100%. In one embodiment, the level of symptoms is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

As used herein, “patient” includes human or non-human (i.e., animal) patient. In a particular embodiment, the invention encompasses both human and nonhuman. In another embodiment, the invention encompasses nonhuman. In another embodiment, the term encompasses human.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, the animal is susceptible to infection by influenza. In some embodiments, an animal may be a transgenic animal, genetically engineered animal, and/or a clone.

As used herein, “therapeutically effective amount” refers to an amount effective, when administered to a human or non-human patient, to provide a therapeutic benefit such as amelioration of symptoms, slowing of disease progression, or prevention of disease. The specific dose of substance administered to obtain a therapeutic benefit will, of course, be determined by the particular circumstances surrounding the case, including, for example, the specific substance administered, the route of administration, the condition being treated, and the individual being treated.

While it is possible for an active ingredient to be administered alone, it may be preferable to present them as pharmaceutical formulations or pharmaceutical compositions as described above. The formulations, both for veterinary/animal and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional biologically active and/or inactive carriers and excipients with or without a biodegradable material, which will be selected in accord with ordinary practice. As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface-active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

The active ingredient can be administered by any route appropriate to the condition. Suitable routes include inhalation, oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In one embodiment, the patient is human.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

In some embodiments, compositions described herein include one or more agents. Exemplary agents include, but are not limited to, small molecules (e.g. cytotoxic agents), nucleic acid (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof.

ILCs, a relatively new cell type, has emerged as an important effector cell at the mucosal surfaces. These cells can be divided into 3 groups, ILC1, ILC2, and ILC3 based on the expression of transcription factors and cytokines. Group 1 ILCs are activated by IL-12, IL-15, and IL-18, and include Natural Killer (NK) cells, which produce Interferon (IFNy). In addition to IFNy expression, NK cells exhibit cytolytic functions which are exerted through granzymes and perforin⁴⁴. ILC2 cells produce IL-4, IL-5, IL-9, IL-13, and amphiregulin, which facilitates tissue repair in the lungs. The ILC3 group contains several subtypes including IL-22-producing natural cytotoxicity receptor (NCR) positive, IL-17 producing NCR negative, and lymphotoxin-expressing Lymphoid Tissue-inducer (LTi) cells. While ILC1 express transcription factor Tbet, ILC2 express RORα, and GATA3 and ILC3 express RORγt. ILCs play a crucial role in regulating immune responses, inflammation, and homeostasis.

Apart from their documented role in immunity to intestinal pathogens, ILCs have been shown to play a role in immune responses against respiratory pathogens such as Klebsiella pneumonia and Streptococcus pneumonia and maintaining epithelial cell integrity following influenza virus infection. The ILC literature is on the biology, activation of ILCs, and role of ILCs in response to pathogens, but whether the activated ILCs confer protection against infectious diseases remained elusive.

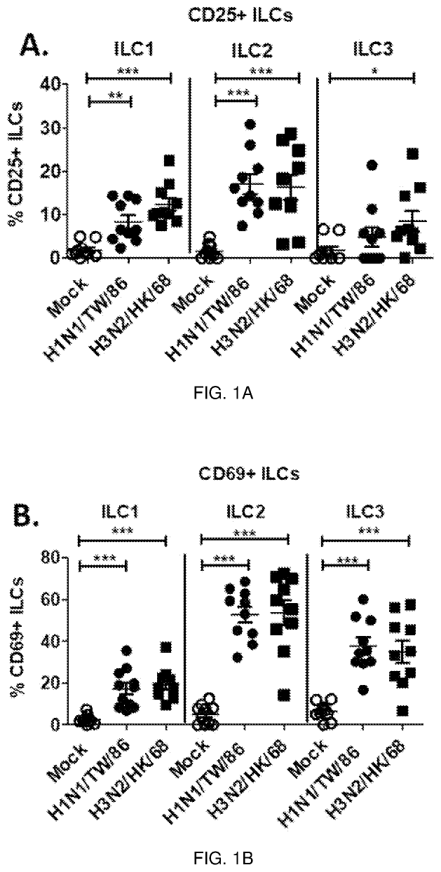

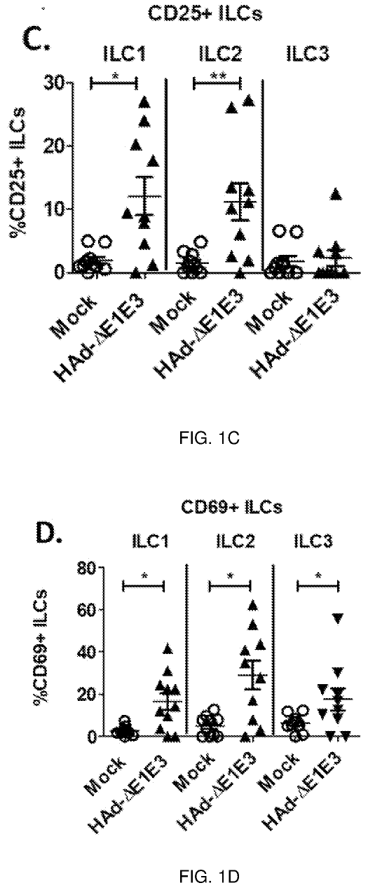

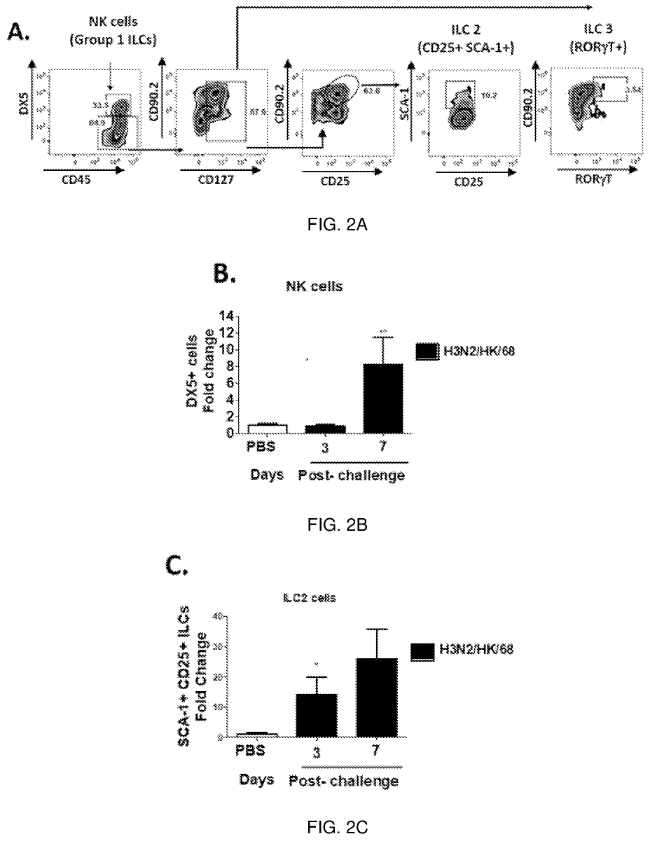

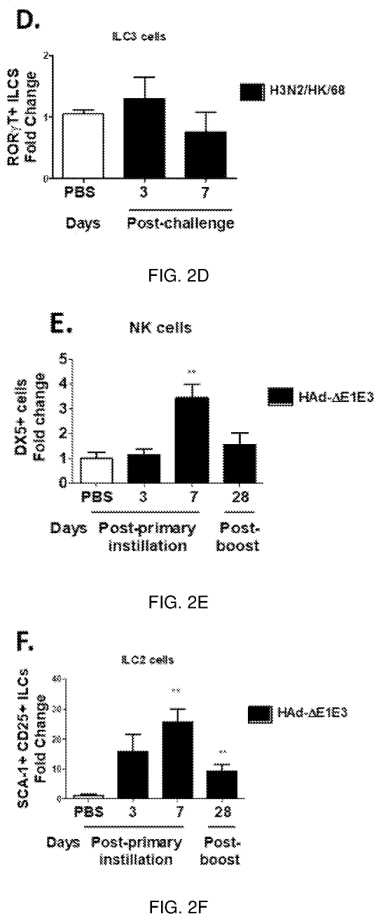

In certain embodiments, the present disclosure provides a potential utility of the activated ILCs to confer protection against a severe influenza epidemic or pandemic situation where a strain-matched vaccine is not available to prevent/contain it as experienced during 2009 pandemic. Besides, the circulation of a drug-resistant epidemic or pandemic influenza further burdens the preventive and control measures. In certain embodiments, human ILC-induced by replicating influenza viruses and HAd-ΔE1E3 were characterized and similar activation profiles of ILC subsets were observed (FIGS. 1A-1D). Robust activation of human ILCs suggests that these cells are important in response to influenza infection in humans. Importantly, HAd-ΔE1E3 also induces a robust activation of human ILCs. These findings were extended from human subjects to a murine model and similar activation of ILCs by influenza virus as well as HAd-ΔE1E3 were demonstrated (FIGS. 2A-2G).

Non-NK group 1 ILCs, (ILC1 and iILC1) have been described in other tissues, but their presence in the lungs as well as specific-markers to identify those cells remains controversial. In certain embodiment, the present disclosure provides that NK cells are found in the lungs and that HAd-ΔE1E3 increased the number of NK cells in the lungs by 7 days post-treatment, however by 28 days post boost, NK cells have returned to baseline. In addition to NK cells, ILC2 cells are also found in the lungs by 3 days post-treatment, which increased to approximately 10-fold and remained at that level up to 4 weeks after the boost. The persistence of ILC2 cells in the lungs suggests that these cells play an important role in conferring protection against subsequent influenza viral challenge.

Group 2 ILCs respond to IL-33, IL-2, IL-25, and TSLP, and express GM-CSF, amphiregulin, IL-5, IL-9 and IL-13^{45,46}. One of the earliest reports on ILCs in influenza infection by Monticelli et al. 2011 described a role for ILCs in pulmonary repair following influenza infection,⁸ suggesting that ILCs promote survival following influenza infection. These cells have now been identified as ILC2 cells, and studies have shown that the tissue repair function is mediated in part by amphiregulin⁷. Later work has shown that suppression of ILC2 cells by IFNy increases susceptibility to influenza infection⁴⁷, further highlighting the potential contribution of ILC2 cells in protection against influenza. HAd-ΔE1E3 also induced ILC3 cells with kinetics similar to those of ILC1-NK cells. ILC3 cells are activated by IL-2, IL-6, IL-23, and IL1β, and express GM-CSF, IL-17, TNF, and lymphotoxin (LT)⁴⁸. Despite a 6-fold increase in ILC3 cells at 7 days post HAd-ΔE1E3 treatment, and at 28 days after HAd-ΔE1E3 boost, ILC3 numbers had returned to baseline. Therefore, it is unlikely that ILC3 cells contributed to protection induced by HAd-ΔE1E3.

It may be impossible to prevent infection during an influenza pandemic until a vaccine homologous to the pandemic virus strain is available; however, limiting disease severity is the key to containing the pandemic and reducing mortality. The 2009 swine flu pandemic resulted in an estimated 60.8 million cases, 274,304 hospitalizations (195,086-402,719), and 12,469 deaths (8,868-18,306) in the United States alone. It took at least 5-6 months for a vaccine matching the pandemic strain to become available⁵⁰, and the global estimates suggest that approximately 201,200 deaths (105,700-395,600) occurred during the first 12 months of the pandemic⁵¹. With the impending threat of an avian influenza pandemic, the importance of developing measures to mitigate influenza-related deaths cannot be overstated⁵². In a pandemic situation, production and distribution of a vaccine promptly are a tremendous challenge⁵³; therefore, approaches that aid in reducing morbidity and facilitate recovery might be a life-saving solution. Intranasal administration of HAd-ΔE1E3 heightened anti-influenza state conferred significant protection against challenge with PR8 virus, and the protection conferred ranged from 100% when challenged 2 days post-treatment to 70% when challenged at 47 days post-treatment⁵⁴. The mechanism of protection was unknown. The potential mechanisms of protection by ILCs and detailed characterization of human and murine ILCs, infiltrating cells, serology, CMI and cytokine and chemokines are addressed in the present disclosure.

In certain embodiments, the present disclosure provides that HAd-ΔE1E3 induced neither serological responses nor CD8⁺ T cell responses against influenza virus. The present disclosure further provides that infiltration of neutrophils, inflammatory monocytes, CD4⁺ and CD8⁺ T cells is consistent with previous observations. Moreover, the present disclosure provides these findings to include interstitial macrophages, CD11b⁺ DCs, and CD103⁺ DCs, which persist up to 28 days post-treatment.

A previous report showed that immune cell recruitment in response to adenoviral vector inoculation is mediated by CXCL10 (IP-10) up to 7 days post-instillation³⁸. The results disclosed herein confirm and these findings were extended to show that the inflammatory response is long-lived as evidenced by the elevation of inflammatory cytokines and chemokine genes weeks after vector administration. These included CXCL10, CXCL9, CCL8, CCL20, and IL12β, which were upregulated at 4 weeks after the boost. The present disclosure highlights the longevity of the cytokine response, which may explain the long-term protection elicited by HAd-ΔE1E3 inoculation strategy. Since these cytokines recruit immune cells to the lungs, they may lead to accelerated innate immune responses that mitigate subsequent influenza infection. Similarly, antiviral genes such as MX-1 and ISG15, which are important in the protection against influenza infection, were also induced by HAd-ΔE1E3, suggesting that they may contribute to protection following influenza challenge. These findings provide support for the concept of activating ILC populations with a non-replicating adenovirus or small molecules during an epidemic or pandemic when a vaccine corresponding to the circulating strain is not available to contain the spread and reduce disease burden.

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLE 1

### Materials And Methods

Generation and characterization of HAd-H7HA and HAd-ΔE1E3 viruses. HAd-H7HA and HAd-ΔE1E3 viruses were constructed as described previously³⁹. Briefly, HAd-H7HA represents HAd-ΔE1E3 expressing the full-length coding region of the HA gene of A/Anhui/1/2013 (H7N9) influenza virus under the control of the cytomegalovirus (CMV) promoter and bovine growth hormone (BGH) polyadenylation signal (polyA). HAd-ΔE1E3 represents the E1, and E3 deleted HAd empty vector. The vectors were plaque purified, and their genomes were analyzed by restriction enzyme digestion and sequencing to confirm the presence or absence of foreign gene cassette. HEK 293 cell monolayers were infected with HAd-ΔE1E3 or HAd-H7HA at a multiplicity of infection (MOI) of 10 PFU per cell and 36 hours post-infection, cells were harvested, and cell lysates were examined for the expression of H7HA protein using a ferret anti-A/Netherland/219/03 (H7N9)-specific antibody by immunoblot analysis³⁹. HAd-ΔE1E3 or HAd-H7HA was grown in 293 cells⁵⁵, purified by cesium chloride density-gradient centrifugation⁵⁵ and titrated by PFU assay in BHH-2C (bovine-human hybrid 2C) cells⁵⁶.

Influenza viruses. Influenza viruses used in these examples include A/Puerto Rico/8/34 (H1N1) [PR8], A/Anhui/1/2013 (H7N9), A/Hong Kong/1/68 (H3N2), A/Taiwan/1/86 (H1N1), and SH2/PR8 (H7N9) containing HA and NA from A/Shanghai/2/2013 (H7N9) and the remaining six gene segments from PR8. Viruses were propagated for 2 days in the allantoic cavity of 10 day-old embryonated chicken eggs. Pooled allantoic fluid was clarified by centrifugation, aliquoted, titrated and stored at −80° C. until use.

Characterization of ILCs in human PBMCs by flow cytometry. Human PBMCs from 10 healthy subjects were isolated by using Vacutainer® CPT™ Mononuclear Cell Preparation Tubes (BD Bioscience, San Jose, Calif.) according to the manufacturer's recommendations. CDC's institutional review board (IRB)-approved written informed consent was obtained from all donors. Cells were incubated with A/Taiwan/1/86 (H1N1) or A/HK/68 (H3N2) at an MOI of 3 PFU per cell for 16 hours or HAd-ΔE1E3 at an MOI of 10 PFU per cell for 16 hours. Cells were then stained with fluorescent antibodies and analyzed by flow cytometry to detect ILCs. The antibody cocktail included CD56-APC, CD3-BV605, CD16-PE, CD127-Pacific Blue, CD117-AmCyan, CRTH2-PECy7, CD69-APC-Cy7, CD25- Alexa Fluor® 700 and a lineage cocktail containing CD8a, CD4, CD14, CD15, CD19, CD20, CD33, CD34, CD203c, and FccRlα conjugated to FITC (Biolegend, San Diego, Calif.).

Animal inoculations and influenza virus challenge. Six to eight-week-old BALB/c mice (Jackson Laboratories, Bar Harbor, Me.) were anesthetized by inhalation of isoflurane and treated by either i.n. or i.m. with HAd-H7HA, HAd-ΔE1E3 or PBS, (5 animals/group). For the i.n. treatment, 50 μl of the influenza viruses or non-replicating adenoviruses was administered into the nostrils, and for the i.m. treatment, 50 μl of the non-replicating adenovirus was injected into each thigh.

For challenge studies, mice were treated as described and given a boost dose 4 weeks post-primary treatment, and then challenged 4 weeks post-boost with 5× lethal dose 50% (5LD₅₀) of wild type A/Anhui/1/2013 (H7N9) or 2LD₅₀ of mouse-adapted PR8 virus. Mice were monitored for weight loss and mortality every day, and mice that lost >25% of their pre-infection body weight were euthanized under anesthesia. Animal research was conducted under the guidance of the CDC's Institutional Animal Care and Use Committee in Association for Assessment and Accreditation of Laboratory Animal Care (AALAC) International-accredited animal facility.

Flow cytometric characterization of cellular infiltrates in the lungs. The lungs were collected at indicated time points post-treatment, and lung homogenates were prepared using the Lung Dissociation Kit and gentleMACS™ Dissociator (Miltenyi Biotec, Auburn, Calif.). Lymphocyte enrichment was performed by density centrifugation using Lymphoprep™ (Stemcell technologies, Cambridge, Mass.). For DC and macrophage identification, cells were stained with anti CD45-AmCyan, MHC II-Alexa 700, CD103 PerCP-Cy5.5, CD64-PE (Biolegend, San Diego, Calif.), CD3-FITC, CD19-FITC, DX5-FITC, CD11c-PECy7, B220-FITC (BD Biosciences, San Jose, Calif.), Mar1-APC, or CD11b Pacific Blue (ebiosciences, San Diego, Calif. and Biolegend, San Diego, Calif.). For ILC identification the following antibodies were used: Lineage-FITC, SCA-1-Pacific Blue, CD45-V500, CD90.2-BV605 (Biolegend), RORγT-PE (BD Biosciences), CD127-PerCP-Cy5.5 (Ebiosciences), CD25-APCCy7 (Biolegend), and DX5-APC (Thermofisher Scientific). For intracellular transcription factor staining, cells were fixed and permeabilized using the FoxP3 transcription factor buffer kit (Biolegend). Samples were analyzed using BD LSRFortessa (BD Biosciences), and the cytometry data were analyzed using FlowJo software (Tree Star, Inc., Ashland, Oreg.).

Gene expression arrays. Lung homogenates were prepared and enriched for lymphocytes as described. RNA was extracted using TRIzol™ (Thermofisher Scientific) and phenol-chloroform extraction method, cleaned using RNA Clean and Concentrator™ (Zymo Research) and then quantified by NanoDrop™ spectrophotometer (Thermo Fisher Scientific). cDNA was prepared using Superscript® III first strand cDNA synthesis kit (Thermofisher Scientific). Quality of cDNA was checked using Qiagen QC control plates, and PCR array and RT-PCR were performed using Qiagen RT² profiler kits for Mouse Antiviral Genes, and Mouse Inflammatory Cytokines and Receptors (Qiagen). qRT PCR was performed using Stratagene 3005p thermocycler. Data were analyzed using Qiagen Software (Qiagen Data Analysis Center, Qiagen GeneGlobal).

Adenoviral DNA detection. DNA from mouse lungs was extracted from lung homogenates using the Qiagen DNeasy Blood and Tissue kit (Qiagen). DNA was quantified using a Nanodrop spectrophotometer (Thermofisher Scientific) and analyzed by qPCR using a set of primer pairs and Taqman® probes specific to the HAd-C5 hexon gene⁵⁷. Genome copies per 50 ng of DNA were quantified using purified HAd-C5 genomic DNA as a standard.

Hemagglutination inhibition assay (HAI). Serum was analyzed by HAI assay using horse RBC as previously described³⁹. Briefly, serum samples from all mice were treated overnight with receptor-destroying enzyme from Vibrio cholerae (Denka Seiken, Tokyo, Japan) at 37° C. to destroy non-specific serum inhibitor activity. Serial dilutions of RDE-treated serum were mixed with 4 hemagglutination units of SH2/PR8 virus for 60 min, followed by addition of 50 μl 1% horse RBC for 60 min. The highest serum dilution inhibiting hemagglutination was taken as the HAI titer.

Influenza micro-neutralization assay. RDE-treated serum was analyzed by micro-neutralization assay using SH2/PR8 virus as previously described³⁹. RDE-treated serum was serially diluted in 96-well plates and incubated with SH2/PR8 viruses at a dose of 2×10³ TCID₅₀/ml for 2 hours at 37° C. MDCK cells were added and incubated overnight. Cells were then fixed with 80% acetone and incubated with biotinylated anti-nucleoprotein antibody (EMD Millipore, Billerica, Mass.), followed by streptavidin-HRP (Southern Biotech, Birmingham, Ala.). Bound HRP was visualized using 1×TMB substrate solution (eBioscience, San Diego, Calif.), and color change was assessed using a microplate reader. The highest serum dilution that generated >50% specific signal was considered as the neutralization titer; 50% specific signal=(OD₄₅₀ virus control−OD₄₅₀ cell control)/2+OD₄₅₀ cell control.

Adenovirus neutralization assay. Mouse serum samples were incubated at 56° C. for 30 min. Serial, two-fold dilutions of each serum sample in 96-well plates were incubated with 100 PFU of HAd-ΔE1E3 for 1 hour at 37° C. followed by addition of 10⁴ BHH-2C⁵⁶ cells in each well. The plates were incubated at 37° C. for 7 days until complete cytopathic effect (c.p.e.) appeared in the virus controls as mentioned previously⁵⁸. The virus neutralizing antibody titer was the reciprocal of the highest serum dilution that completely prevented the development of c.p.e.

ELISA. ELISA was performed to detect H7HA- or H7N9 virus-specific IgG antibody levels in the serum. Briefly, Immunol plates (Thermofisher Scientific) were coated overnight with 1 μg/m1 of H7HA or 50 HAU of SH2/PR8 virus at 4° C. and then blocked for 1 hour with PBS/0.05%Tween-20 (PBST) containing 4% BSA at room temperature. Serum was serially diluted in PBST and incubated with antigen-coated plates for 2 hours at room temperature. After washing with PBST, wells were probed with HRP anti-mouse IgG for 1 hour at room temperature. Signal was developed using 1×TMB substrate solution (eBioscience), and color change was detected using a BioTek microplate reader.

Statistical analysis. Statistical analyses were performed using GraphPad Prism 5.0 software (GraphPad Software, La Jolla, Calif.), and groups were compared by Mann-Whitney U test with a p-value of ≤0.05 is considered statistically significant. Data are presented as mean±SEM.

### EXAMPLE 2

### Activation of ILCs From Human Peripheral Blood Monocytes Cells (PBMCs) with Influenza or HAd-ΔE1E3 In Vitro

To activate ILCs, PBMCs purified from 10 healthy donors were incubated with A/Taiwan/1/86 (H1N1) or A/Hong Kong/1/68 (H3N2) influenza virus at a multiplicity of infection (MOI) of 3 plaque-forming units (PFU) per cell for 16 hours and phenotypic analyses were performed by flow cytometry to detect activation of ILCs. ILCs can be identified by lack of lineage (lin) markers (CD8α, CD4, CD14, CD15, CD19, CD20, CD33, CD34, CD203c, and F_{Cε}Rlα) and expression of a combination of cell surface markers. ILC1 cells were identified as lin⁻ CD127⁺, cKIT⁻ and CRTH2⁻, ILC2 cells as lin⁻ CD127⁺ CRTH2⁺, and ILC3 as lin⁻ CD127⁺ cKIT⁺ by adopting a proposed gating strategy for uniform immune-phenotyping of human ILCs²². ILCS activation was detected by upregulation of activation markers CD69 and CD25²³.

H1N1 or H3N2 influenza virus infection increased CD25 expression on ILC1 cells to approximately 4-6 folds as compared to uninfected cells. A similar increase was observed in CD69 expression (FIG. 1A). Significant upregulation of both CD25 (10-fold) and CD69 (10-fold) was detected on ILC2 cells upon infection with H1N1 and H3N2 influenza viruses (FIG. 1B). Interestingly, H3N2 but not H1N1 infection significantly increased CD25 expression in ILC3 cells (FIG. 1A); however, CD69 was increased 6-fold by both viruses as compared to uninfected cells (FIG. 1B). An increase in CD69 expression on Natural Killer (NK) cells was also observed (data not shown) which is consistent with previous reports that influenza virus induces activation of NK cells²⁴.

Next, it was sought to determine whether HAdΔ-E1E3 can induce activation of ILCs. Human PBMCs were treated with HAdΔ-E1E3 at an MOI of 10 PFU per cell for 16 hours. HAdΔ-E1E3 also induced upregulation of CD25 in ILC1 and ILC2 cells but not ILC3 (FIG. 1C). ILC1, ILC2 and ILC3 cells significantly increased the expression of CD69 in response to HAdΔ-E1E3 treatment (FIG. 1D), suggesting that adenovirus activates human ILCs.

### EXAMPLE 3

### Influenza and HAd-ΔE1E3 Viruses Activate ILCs in the Lungs of Mice In Vivo

Having found that an influenza virus or adenovirus can activate human ILCs, the induction of ILCs was further investigated in a mouse model. The extent to which influenza virus or HAd-ΔE1E3 affects the ILC population in the lungs is unclear. Hence, to determine the effect of influenza on lung ILCs, BALB/c mice were given 20 mouse infectious dose 50 (MID₅₀) of A/HK/68 (H3N2) or PBS via the intranasal (i.n.) route and the lungs were harvested at 3 and 7 days postinfection to determine changes in ILCs by flow cytometry. Group 1 ILC (NK cells), which were identified by expression of DX5, were not induced at day 3 but increased 8-fold by day 7 (FIG. 2B). ILC2 cells were identified by CD90.2, CD127, CD25, and SCA-1 expression. By 3 days post-infection, ILC2 cells increased by approximately 15-fold over the PBS control group, which further increased to 25-fold at day 7 (FIG. 2C). These data were consistent with previous reports that influenza infection induces NK cells and ILC2 cells in the lungs. ILC3 cells, identified by CD90.2, CD127, and RORγT expression, did not increase with influenza infection (FIG. 2D).

To investigate the effect of adenovirus on ILCs in the lungs, mice were treated with 5×10⁷ PFU of HAd-ΔE1E3 or PBS via i.n. route, and ILCs were measured in the lungs by flow cytometry. Similar to the response to influenza, group 1 ILC (NK cells), were not induced at day 3 but were increased 3-fold by day 7 (FIG. 2E). By 3 days post-infection, induction of ILC2 cells in the HAd-ΔE1E3 group was approximately 15-fold compared to the PBS control group, which further increased to 25-fold at day 7 (FIG. 2F). In contrast to the H3N2 influenza virus, HAd-ΔE1E3 induced a 6-fold increase in ILC3 cells by day 7 (FIG. 2G).

Having observed that HAd-ΔE1E3 induces ILCs early in the mouse lungs, it was sought to determine the longevity of the response, and the extent to which re-administration would affect the ILC response in the lungs. Mice were treated with HAd-ΔE1E3 via i.n route, and after 4 weeks, a second dose was administered. Four weeks after the second treatment, ILCs in the lungs were assessed by flow cytometry. At 4 weeks after the boost, NK cells were no longer elevated in the lungs (FIG. 2E); however, ILC2 cells remained upregulated approximately 8-fold over the PBS control group (FIG. 2F). ILC3 cells remained unchanged 4 weeks after the boost (FIG. 2G). These data suggest that HAd-ΔE1E3 induces a sustained induction of ILC2 cells in the lungs.

### EXAMPLE 4

### Characterization of Innate Immune Responses in the Lungs

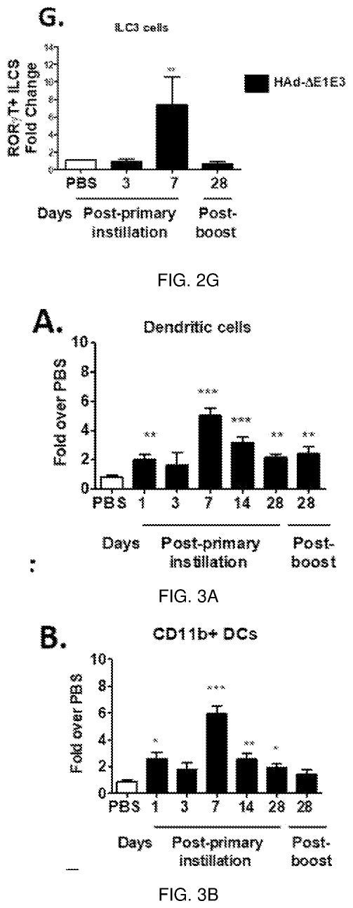

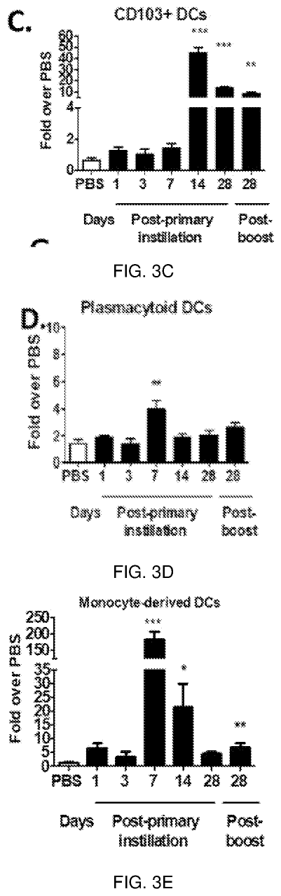

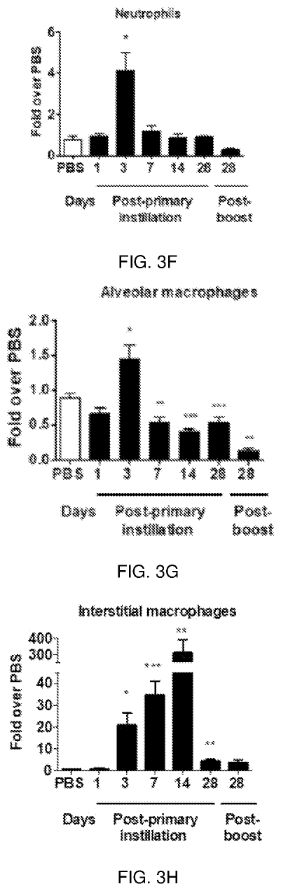

cellular infiltrates were characterized in the lungs of mice after a single dose as well as following a boost with HAd-ΔE1E3. By day 1 post-treatment, the overall DC population increased by 2-fold, and the response reached a 5-fold at 7 days post-treatment, which was the peak of the response (FIG. 3A). Even at 28 days following the boost, the number of DCs remained at 2-fold above the PBS control group. Several subsets of DCs are induced in the lungs, each with a specialized role²⁵. Plasmacytoid DCs and those of the myeloid lineage were identified. Plasmacytoid DCs, a relatively small but potent type I interferon (IFN)-producing population²⁶ increased 4-fold in response to the treatment, with a peak at 7 days (FIG. 3D). CD11b⁺ DCs comprise a majority of the DCs within the myeloid DC subset in the lungs^{27,28}, which express distinct pathogen-recognition receptors and are critical for antigen presentation to CD8⁺ T cells^{29,30}. It was found that CD11b⁺ DCs increased 6-fold compared to the PBS group at 28 days post-primary treatment. No further increase was detected in CD11b⁺ DCs 28 days after the boost.

Another subset analyzed was CD103⁺ DCs, which work in concert with CD111b⁺ DCs to generate optimal CD4⁺ and CD8⁺ T cell responses^{27,31}. Impressively CD103⁺ DCs increased over 40-fold compared to the PBS group at 14 days post-treatment (FIG. 3C). This population remained elevated 15-fold at 28 days post-treatment and 8-fold after the mice received a boost suggesting that HAd-ΔE1E3 selectively results in the sustained accumulation of CD103⁺ DCs in the lungs.

In response to viral infection, monocytes from the peripheral blood migrate to the lungs where they differentiate into DCs and macrophages³². an increase in monocyte-derived inflammatory DCs was detected, which peak at 7 days post-treatment. This inflammatory DC response decreased after the peak; however, a 5-fold increase above the PBS group was observed at 28 days (FIG. 3E). After the boost, this population remained 5-fold elevated above the PBS group. Overall, these data suggest that HAd-ΔE1E3 alters the composition of DCs in the lungs. Interestingly, CD11b⁺ DCs are the most abundant subset during the acute response, and CD103⁺ DCs are the predominant subtype at the time of influenza challenge suggesting differential roles for these cells during the immune response.

Alveolar macrophages are also important, as they have been shown to phagocytose the non-replicating adenovirus in the respiratory tract³³ and induce inflammatory cytokines³⁴. After an initial 3-fold increase in the number of alveolar macrophages in the lungs, significant decreases were observed between 7 and 28 days in treated mice compared to the PBS group. This cell type remained low in the lungs despite the booster treatment. Although having been identified years ago, little is known regarding interstitial macrophages and their contribution to the inflammatory response to adenovirus infection. HAd-ΔE1E3 induced accumulation of interstitial macrophages beginning 3 days post-treatment, which continued to increase until 14 days when a 300-fold increase was detected (FIG. 3H). This population was not significantly changed at 28 days post-boost. Further, an influx of neutrophils was observed marked by a 4-fold increase by 3 days post-treatment (FIG. 3F).

### EXAMPLE 5

### Induction of Cytokines, Chemokines and Antiviral Factors in the Lungs

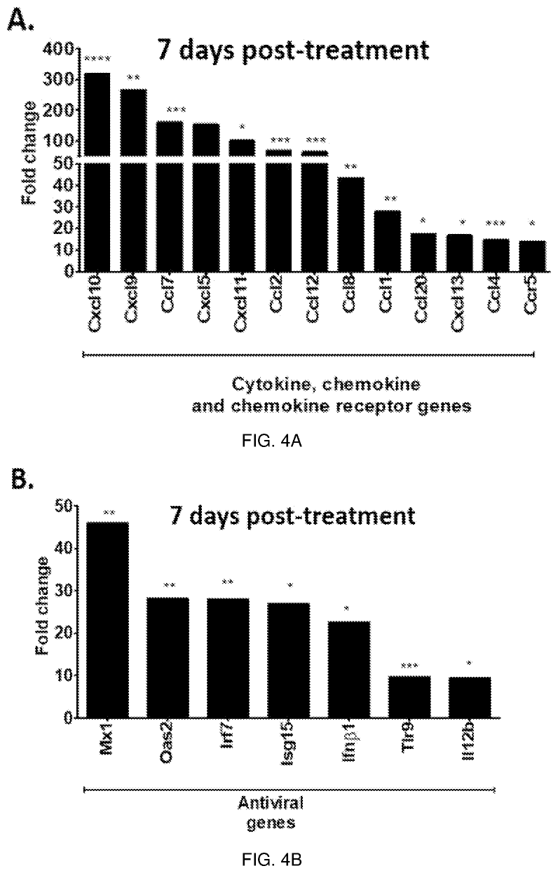

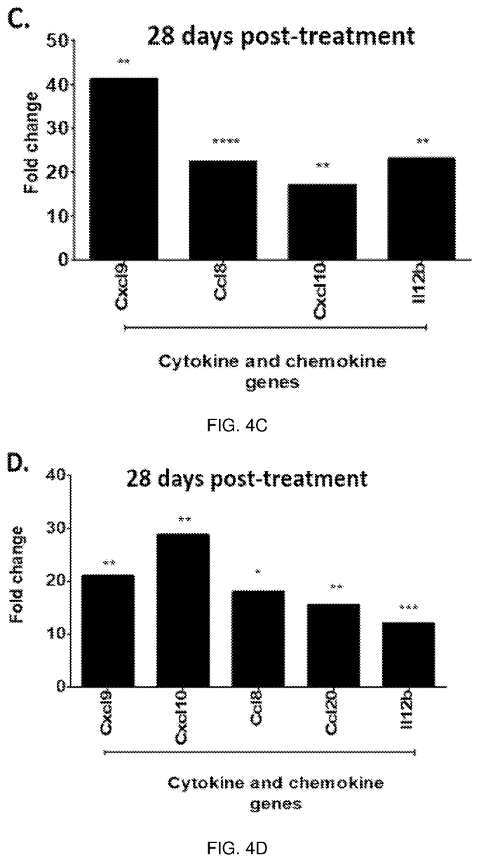

Sensing of the virus capsid by endosomal TLR9, and recognition of adenovirus DNA by the sensor cGAS initiates signaling with ultimately activates the MAP Kinase, NF-kB, IRF3 and NLR family pyrin domain containing 3 (NLRP3) signaling cascades³⁵. These pathways combined result in a powerful inflammatory response in the lungs (FIGS. 4A-4D). Cytokines such as IL6, TNFα, CXCL10, and CXCL9 are hallmark features of this response following systemic administration of non-replicating adenoviruses^{36,37}; however, studies focusing on the lung response following intranasal administration have been limited. Specifically, a systematic investigation of the inflammatory and antiviral genes that may protect adenovirus-treated mice against other respiratory viruses such as influenza has not been reported³⁴.

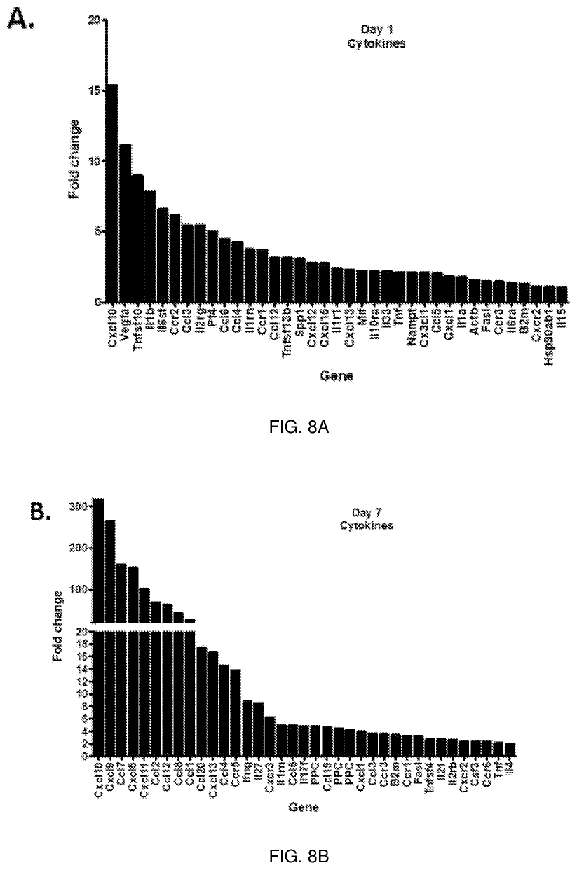

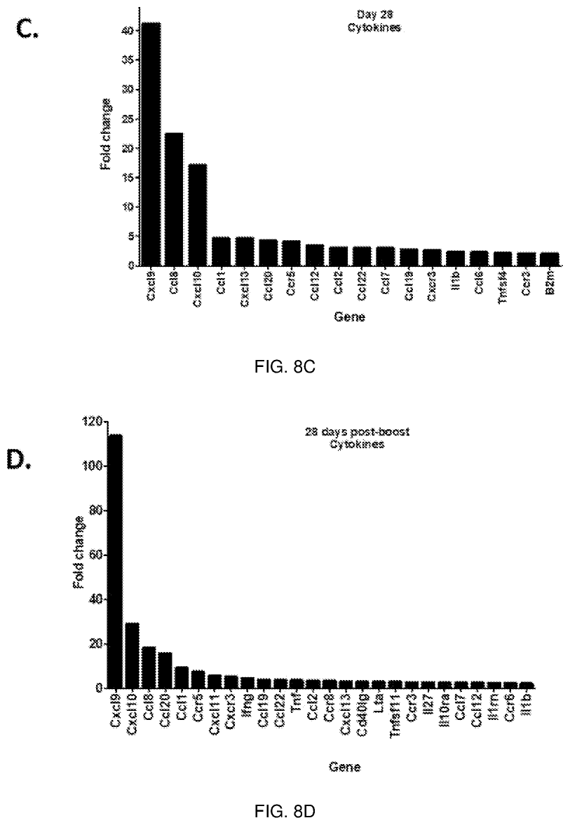

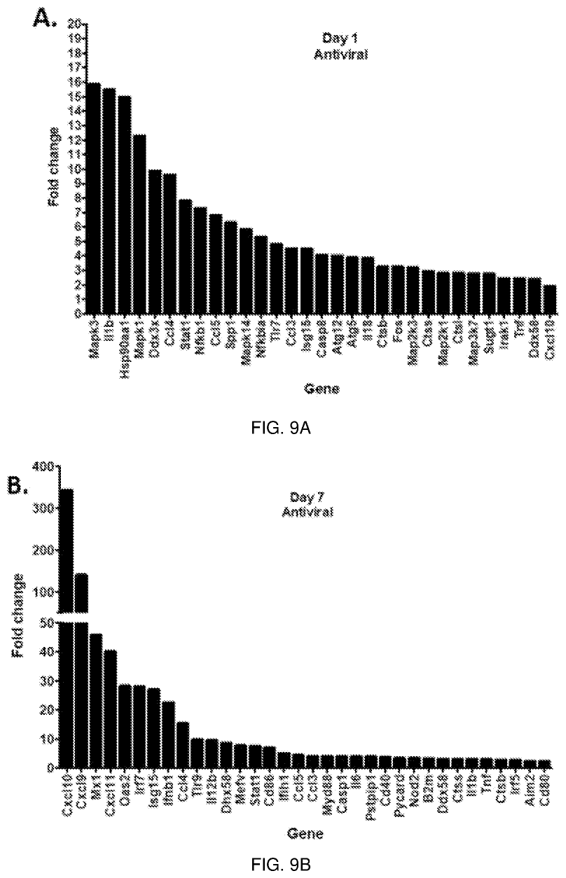

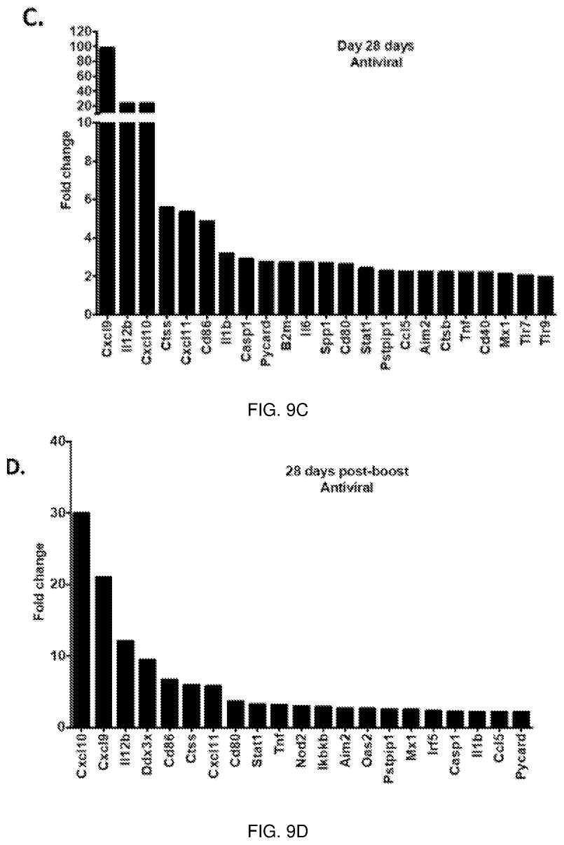

For a comprehensive analysis of the magnitude of the inflammatory response, qPCR arrays were used to determine the effect of i.n. administration of HAd-ΔE1E3 on the induction of genes encoding inflammatory cytokines and their receptors, as well as antiviral proteins that may contribute to protection against influenza. By day 1 post-administration, a 15-fold induction of CXCL10 was detected (FIGS. 8A-8D), which is consistent with previous reports of early induction of this chemokine in the lungs in response to adenoviral vector administration³⁸. Between 5- and 8-fold induction of CCL3, CCL6, and receptors CCR2 and IL2 receptor γ were also observed (FIGS. 8A-8D). Among the highly induced cytokines were VEGFαwith 11-fold upregulation, Tnfsf10 of the TNF-alpha superfamily with over 8-fold induction, IL1β, IL6st- a signal transducer in the IL-6 pathway and CCL4.

At day 7 post-treatment, a more robust response was detected (FIGS. 4A and 4B). Chemotaxins CXCL10, CXCL9, CCL7 CXCL11, and CXCL5 were elevated, with CXCL9 and CXCL10 (IP-10) having the highest levels of induction of 170- fold and 300-fold upregulation, respectively (FIG. 4B). A plethora of other antiviral genes was also induced, including IL12β, which can activate NK cells (FIG. 2E). In addition to these, many antiviral genes relevant in the response against influenza were upregulated. Specifically, MX-1, OAS2, IRF7, ISG15, IRF7, STAT1, and IFN-β, all indicative of activation of the type I IFN pathway, in addition to the inflammatory pathways (FIGS. 4A and 4B).

By 28 days post-boost, a majority of innate immune infiltrates had subsided (FIG. 4); however, ILC2 cells and CD103⁺ DCs remained elevated. At that same time point, CXCL9, CXCL10, CCL8, CXCL10 CCI20, and IL12β remained significantly elevated, with a 20-fold increase in CXCL9, 18-fold induction in CCL8, 15-fold induction in CCL20 and 12-fold induction in IL12β in the adenovirus-treated group compared to the PBS group (FIG. 4D). Overall, these data show that the global changes in the inflammatory response extend beyond what was known and that changes are sustained for weeks after administration, suggesting that HAd-ΔE1E3 administration induces a long-lived inflammatory state composed of elevated cytokines as well as innate lymphoid cells that persists up to 4 weeks after the boost.

### EXAMPLE 6

### Activated ILCs Protect against Lethal Influenza Infection

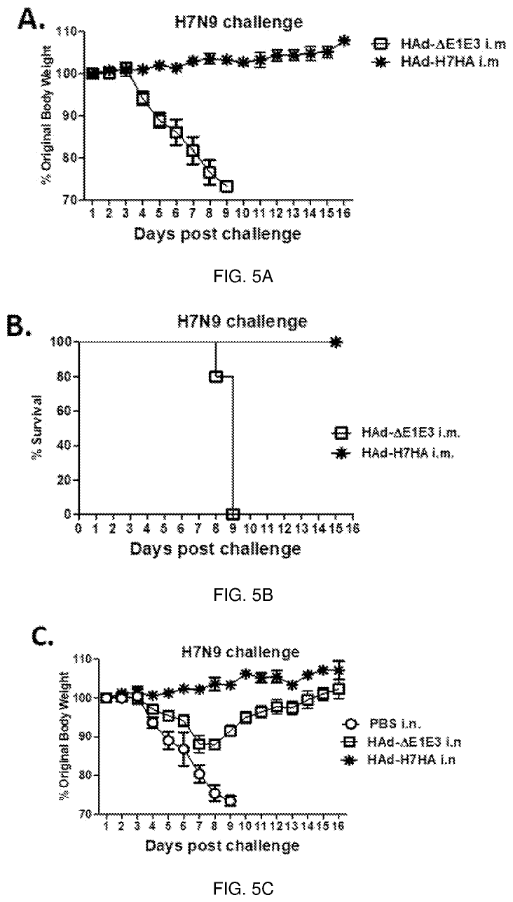

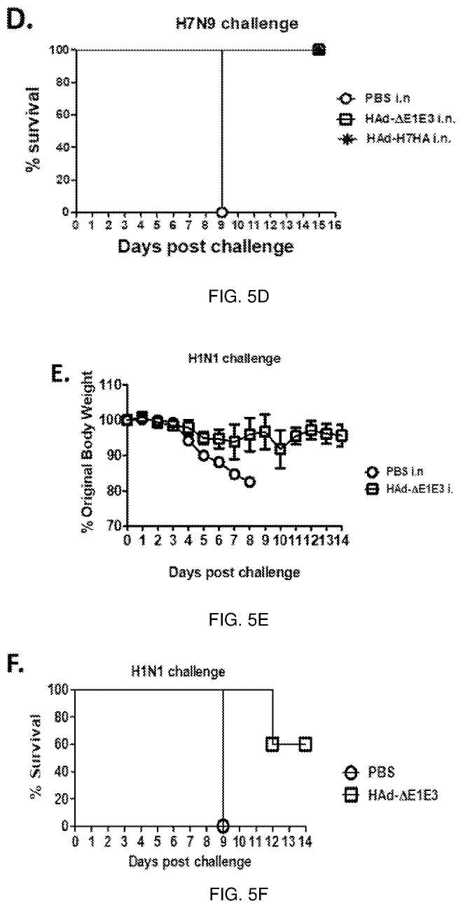

To investigate the effect of this sustained ILC response elicited by non-replicating adenovirus in the lungs on the response to influenza challenge, BALB/c mice were treated either i.m. or i.n. with HAd-ΔE1E3 or non-replicating adenovirus expressing H7HA (HAd-H7HA) and boosted after four weeks. Four weeks after the boost, the mice were challenged with a 5LD₅₀ dose of /Anhui/2013 (H7N9) influenza virus. Mice that were immunized i.m. with HAd-H7HA were 100% protected from the influenza H7N9 challenge (FIG. 5A); however, mice that received HAd-ΔE1E3 i.m. succumbed to the challenge, with 100% mortality by day 9 post-challenge, consistent with the previous work³⁹ (FIG. 5B). Mice treated i.n. with HAd-ΔE1E3 although lost weight following challenge, but 100% of the mice survived (FIGS. 5C and 5D).

These data suggested that activation of ILCs locally but not systemically confers protection against the challenge. To determine whether the ILC response elicited by adenovirus extends to other influenza strains, mice were challenged with 2LD₅₀ of mouse-adapted influenza A/PR8/34 (H1N1) and it was found that ILC activation resulted in 60% survival. These data suggest that the ILC response and other innate immune cells elicited by adenovirus treatment promote survival following lethal influenza challenge.

### EXAMPLE 7

### HAd-ΔE1E3-Induced Protection against Influenza is in the Absence of Influenza-Specific Antibody Responses

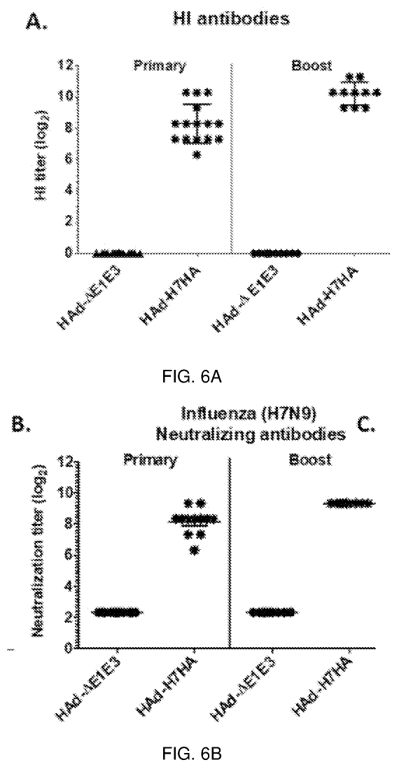

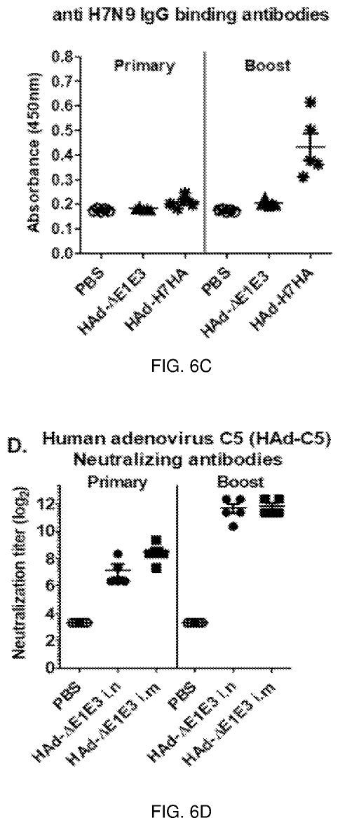

Because the antibody response is critical for protection against influenza,^{39,40} the ability of HAd-ΔE1E3 or HAd-H7HA to induce protective antibodies against influenza H7N9 was investigated. Mice were treated i.n. with HAd-ΔE1E3 or HAd-H7HA, bled 3 weeks post-primary and post-booster inoculations and serum samples were analyzed for hemagglutination inhibiting (HI) antibodies (FIG. 6A) against SH2/PR8 (H7N9) virus. Mice treated with HAd-H7HA elicited HI antibodies after primary treatment, whereas HAd-ΔE1 E3-treated mice failed to induce HI antibodies even after the boost. Similarly, HAd-H7HA elicited H7N9-neutralizing antibodies while HAd-ΔE1E3 did not (FIG. 6B). Both HAd-ΔE1E3 and HAd-H7HA induced similar levels of adenovirus-neutralizing antibodies, regardless of the route of treatment (FIG. 6D).

Next, the serum samples were tested for non-neutralizing yet cross-reactive antibodies, since they can contribute to protection against SH2/PR8 (H7N9)⁴¹. Sera from mice treated with PBS, HAd-ΔE1E3, or HAd-H7HA were tested for H7N9-specific binding antibodies using ELISA. It was found that HAd-ΔE1E3 does not induce H7N9-specific IgG antibodies (FIG. 6C). Altogether, the serological data suggest that with the protection mediated by HAd-ΔE1E3 against influenza H7N9 challenge was not due to H7-specific cross-reactive antibodies.

### EXAMPLE 8

### HAd-ΔE1E3 Does Not Induce Cross-Reactive CD8 T Cells against Influenza Nucleoprotein (NP), a Major CD8 T Cell Target

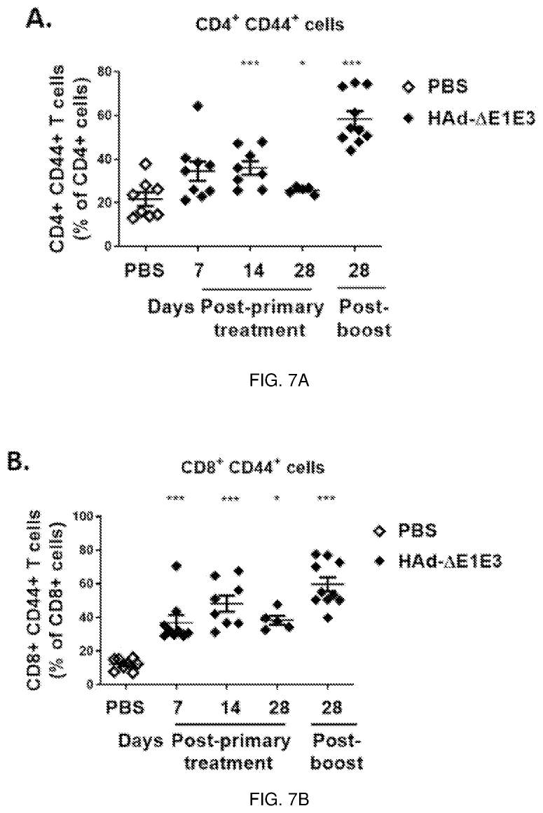

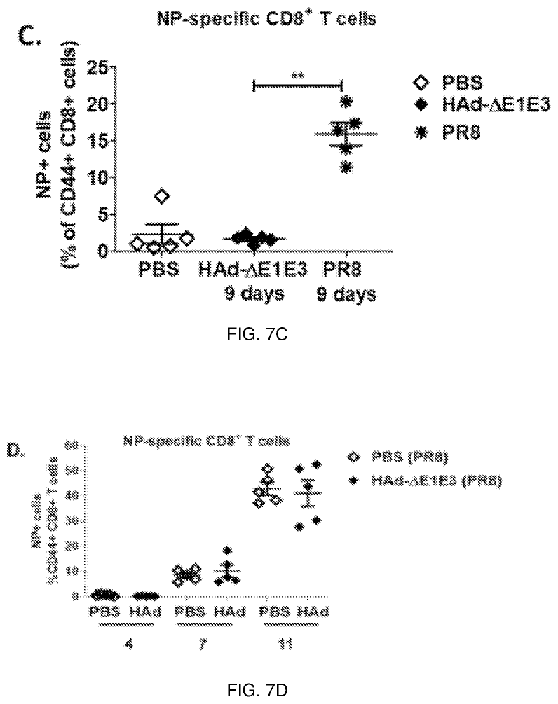

It was investigated if HAd-ΔE1E3 treatment induces activated T cells in the lungs, which may protect against influenza infection. Mice were treated with 5×10⁷ PFU of HAd-ΔE1E3 or PBS and boosted at 4 weeks after primary treatment. Lungs were collected at 7, 14 and 28 days after primary inoculation, and 28 days after the boost, the same time point at which mice had been challenged with H7N9 or H1N1 virus (FIGS. 5A-5F). CD4⁺ and CD8⁺ T cell activation were determined by CD44 expression. Percentage of activated CD4⁺ T cells significantly increased at 14 days (36%), and although they had decreased by 28 days (27%), the response remained above baseline (23%). Interestingly, at 28 days post-boost, approximately 50% of all CD4⁺ T cells remained activated (FIG. 7A).

A significant increase in activated CD8⁺ T cells was detected as early as 7 days post-treatment with 35% of CD8⁺ T cells being CD44⁺ compared to 13% in PBS-treated mice. In contrast to CD4⁺ T cells, the number of activated CD8⁺ T cells remained elevated at 28 days post-primary treatment (37%) (FIG. 7B). The status of the CD8⁺ T cells was also determined 28 days after the boost and it was found that 55% of CD8⁺ T cells were activated at this time point.

The conserved NP of influenza A virus is a major target of immunodominant CD8⁺ T-cell responses²⁵. It was tested whether HAd-ΔE1E3 induced cross-reactive CD8⁺ T cells against influenza NP. Mice were treated i.n. with PBS, 5×10⁷ PFU of HAd-ΔE1E3 or 20MID₅₀ PR8 and harvested 9 days post-treatment. Mice inoculated with H1N1 induced NP-specific CD8⁺ T cells; however, no NP-specific CD8 T cells were detected in mice treated with HAd-ΔE1E3 (FIG. 7C). Further, it was also tested whether administration of HAd-ΔE1E3 alters the kinetics and magnitude of the influenza-specific CD8⁺ T cell responses, thereby giving treated mice a survival advantage compared to untreated mice. Mice were treated with HAd-ΔE1E3 or PBS, and 4 weeks after boost were challenged with 20MID₅₀ of PR8 virus, and CD8⁺ T cells were measured at 4, 7 and 11 days after challenge. HAd-ΔE1E3 treatment did not affect the kinetics or magnitude of the influenza-specific responses (FIG. 7D.) Altogether, these data suggest that HAd-ΔE1E3 results in sustained T cell activation in the lungs but does not induce cross-reactive influenza-specific T cells against NP that could protect against influenza infection.

### EXAMPLE 9

### Persistence of HAd-ΔE1E3

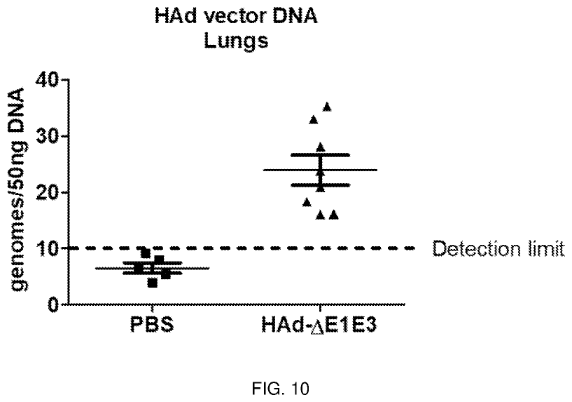

Because the protective immune response was elicited by HAd-ΔE1E3, it was important to determine whether viral persistence in the lungs contribute to the observed sustained ILCs and inflammatory responses. Previous studies have shown that the bulk of adenoviral DNA is eliminated from the liver and other tissues within days of intravenous infusion⁴². Other studies have shown that non-replicating adenoviruses can persist in muscle following intramuscular injection and that vector presence drives persistence of activated antigen-specific CD8⁺ T cells⁴³. Here, the presence of adenovirus genomes was observed at the time of influenza viral challenge (28 days post-boost), by qPCR using primers for the hexon gene. Adenovirus DNA was detectable at approximately 25 genomes per 50 nanograms of DNA (FIG. 10), suggesting that low-level persistence may contribute to the sustained ILCs and innate immune responses including elevated levels of CXCL9, CXCL10, CCI8, and IL12β observed up to 28 days after the boost.

The present disclosure for the first time provides that HAd-ΔE1E3 induces diverse and robust induction of all three ILC populations in the lungs. While much of the inflammatory response subsides after the initial acute response, ILC2 cells and several cytokines remain elevated 4 weeks after the boost. The data presented in the present disclosure show that ILCs protect against influenza viral challenge. In a severe epidemic with a variant virus or pandemic situation, administering the population with HAd-ΔE1E3 to induce ILCs may lower mortality and prevent the virus spread until a strain-matched vaccine is available.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Vivier E, Artis D, Colonna M, et al. Innate Lymphoid Cells: 10 Years On. Cell 2018; 174(5): 1054-66.
2. Lai D M, Shu Q, Fan J. The origin and role of innate lymphoid cells in the lung. Mil Med Res 2016; 3: 25.
3. Crome S Q, Ohashi P S. Immunoregulatory functions of innate lymphoid cells. J Immunother Cancer 2018; 6(1): 121.
4. Crome S Q, Nguyen L T, Lopez-Verges S, et al. A distinct innate lymphoid cell population regulates tumor-associated T cells. Nat Med 2017; 23(3): 368-75.
5. Salome B, Jandus C. Innate lymphoid cells in antitumor immunity. J Leukoc Biol 2018; 103(3): 479-83.
6. Simoni Y, Fehlings M, Kloverpris H N, et al. Human Innate Lymphoid Cell Subsets Possess Tissue-Type Based Heterogeneity in Phenotype and Frequency. Immunity 2017; 46(1): 148-61.
7. Colonna M. Innate Lymphoid Cells: Diversity, Plasticity, and Unique Functions in Immunity. Immunity 2018; 48(6): 1104-17.
8. Monticelli L A, Sonnenberg G F, Abt M C, et al. Innate lymphoid cells promote lung-tissue homeostasis after infection with influenza virus. Nat Immunol 2011; 12(11): 1045-54.
9. Globinska A, Kowalski M L. Innate lymphoid cells: the role in respiratory infections and lung tissue damage. Expert Rev Clin Immunol 2017; 13(10): 991-9.
10. Vashist N, Trittel S, Ebensen T, Chambers B J, Guzman C A, Riese P. Influenza-Activated ILC1s Contribute to Antiviral Immunity Partially Influenced by Differential GITR Expression. Front Immunol 2018; 9: 505.
11. Luliano A D, Roguski K M, Chang H H, et al. Estimates of global seasonal influenza-associated respiratory mortality: a modelling study. Lancet 2018; 391(10127): 1285-300.
12. World Health O. Influenza (Seasonal). 2018.
13. Yiu Lai K, Wing Yiu Ng G, Fai Wong K, et al. Human H7N9 avian influenza virus infection: a review and pandemic risk assessment. Emerg Microbes Infect 2013; 2(8): e48.
14. Jhung M A, Epperson S, Biggerstaff M, et al. Outbreak of variant influenza A(H3N2) virus in the United States. Clin Infect Dis 2013; 57(12): 1703-12.
15. de Jong M D, Tran T T, Truong H K, et al. Oseltamivir resistance during treatment of influenza A (H5N1) infection. N Engl J Med 2005; 353(25): 2667-72.
16. Deyde V M, Xu X, Bright R A, et al. Surveillance of resistance to adamantanes among influenza A(H3N2) and A(H1 N1) viruses isolated worldwide. J Infect Dis 2007; 196(2): 249-57.
17. Drinka P J, Haupt T. Emergence of rimantadine-resistant virus within 6 days of starting rimantadine prophylaxis with oseltamivir treatment of symptomatic cases. J Am Geriatr Soc 2007; 55(6): 923-6.
18. He G, Qiao J, Dong C, He C, Zhao L, Tian Y. Amantadine-resistance among H5N1 avian influenza viruses isolated in Northern China. Antiviral Res 2008; 77(1): 72-6.
19. Shobugawa Y, Saito R, Sato I, et al. Recurrence and persistence of fever in children who developed amantadine-resistant influenza viruses after treatment. Tohoku J Exp Med 2008; 214(2): 129-38.
20. Administration USFaD. FDA approves new drug to treat influenza 2018.
21. Schnirring L. Experts on watch for resistance to new flu drug. Jan. 18 2019 2019.
22. Hardman C S, Chen Y L, Salimi M, et al. CD1a presentation of endogenous antigens by group 2 innate lymphoid cells. Sci Immunol 2017; 2(18).
23. Salimi M, Wang R, Yao X, et al. Activated innate lymphoid cell populations accumulate in human tumour tissues. BMC Cancer 2018; 18(1): 341.
24. Jost S, Quillay H, Reardon J, et al. Changes in cytokine levels and NK cell activation associated with influenza. PloS one 2011; 6(9): e25060.
25. Yewdell J W, Bennink J R, Smith G L, Moss B. Influenza A virus nucleoprotein is a major target antigen for cross-reactive anti-influenza A virus cytotoxic T lymphocytes. Proc Natl Acad Sci USA 1985; 82(6): 1785-9.
26. Guilliams M, Lambrecht B N, Hammad H. Division of labor between lung dendritic cells and macrophages in the defense against pulmonary infections. Mucosal Immunol 2013; 6(3): 464-73.
27. Lambrecht B N, Hammad H. Lung dendritic cells in respiratory viral infection and asthma: from protection to immunopathology. Annu Rev Immunol 2012; 30: 243-70.
28. Plantinga M, Guilliams M, Vanheerswynghels M, et al. Conventional and monocyte-derived CD11b(+) dendritic cells initiate and maintain T helper 2 cell-mediated immunity to house dust mite allergen. Immunity 2013; 38(2): 322-35.
29. Guilliams M, Ginhoux F, Jakubzick C, et al. Dendritic cells, monocytes and macrophages: a unified nomenclature based on ontogeny. Nat Rev Immunol 2014; 14(8): 571-8.
30. Merad M, Sathe P, Helft J, Miller J, Mortha A. The dendritic cell lineage: ontogeny and function of dendritic cells and their subsets in the steady state and the inflamed setting. Annu Rev Immunol 2013; 31: 563-604.
31. Ho A W, Prabhu N, Betts R J, et al. Lung CD103+dendritic cells efficiently transport influenza virus to the lymph node and load viral antigen onto MHC class I for presentation to CD8 T cells. J Immunol 2011; 187(11): 6011-21.
32. Rodero M P, Poupel L, Loyher P L, et al. Immune surveillance of the lung by migrating tissue monocytes. Elife 2015; 4: e07847.
33. Worgall S, Leopold P, Wolff G, Ferris B, Van Rooijen N, G. Crystal R. Role of Alveolar Macrophages in Rapid Elimination of Adenovirus Vectors Administered to the Epithelial Surface of the Respiratory Tract; 1997.
34. Zsengeller Z, Otake K, Hossain S A, Berclaz P Y, Trapnell B C. Internalization of adenovirus by alveolar macrophages initiates early proinflammatory signaling during acute respiratory tract infection. Journal of Virology 2000; 74(20): 9655-67.
35. Gregory S M, Nazir S A, Metcalf J P. Implications of the innate immune response to adenovirus and adenoviral vectors. Future Virol 2011; 6(3): 357-74.
36. Shayakhmetov SAaDM. Innate Immune Response to Adenovirus Vector Administration In Vivo. Adenoviral Vectors for Gene Therapy Elsevier Inc.; 2016: Pages 349-66.
37. Thaci B, Ulasov I V, Wainwright D A, Lesniak M S. The challenge for gene therapy: innate immune response to adenoviruses. Oncotarget 2011; 2(3): 113-21.
38. Zeng X, Moore T A, Newstead M W, Deng J C, Lukacs N W, Standiford T J. IP-10 mediates selective mononuclear cell accumulation and activation in response to intrapulmonary transgenic expression and during adenovirus-induced pulmonary inflammation. J Interferon Cytokine Res 2005; 25(2): 103-12.
39. Cao W, Liepkalns J S, Hassan A O, et al. A highly immunogenic vaccine against A/H7N9 influenza virus. Vaccine 2016; 34(6): 744-9.
40. Wang X, Fu W, Yuan S, et al. Both haemagglutinin-specific antibody and T cell responses induced by a chimpanzee adenoviral vaccine confer protection against influenza H7N9 viral challenge. Sci Rep 2017; 7(1): 1854.
41. Henry Dunand C J, Leon PE, Huang M, et al. Both Neutralizing and Non-Neutralizing Human H7N9 Influenza Vaccine-Induced Monoclonal Antibodies Confer Protection. Cell Host Microbe 2016; 19(6): 800-13.
42. Worgall S, Wolff G, Falck-Pedersen E, Crystal R G. Innate immune mechanisms dominate elimination of adenoviral vectors following in vivo administration. Hum Gene Ther 1997; 8(1): 37-44.
43. Tatsis N, Fitzgerald J C, Reyes-Sandoval A, et al. Adenoviral vectors persist in vivo and maintain activated CD8+ T cells: implications for their use as vaccines. Blood 2007; 110(6): 1916-23.
44. Cyril Seillet G T B, Nicholas D. Huntington. Development, Homeostasis, and Heterogeneity of NK Cells and ILC1. Current Topics in Microbiology and Immunology 2015; 395: 37-61.
45. Camelo A, Rosignoli G, Ohne Y, et al. IL-33, IL-25, and TSLP induce a distinct phenotypic and activation profile in human type 2 innate lymphoid cells. Blood Adv 2017; 1(10): 577-89.
46. Li B W S, Stadhouders R, de Bruijn M J W, et al. Group 2 Innate Lymphoid Cells Exhibit a Dynamic Phenotype in Allergic Airway Inflammation. Front Immunol 2017; 8: 1684.
47. Califano D, Furuya Y, Roberts S, Avram D, McKenzie A N J, Metzger D W. IFN-gamma increases susceptibility to influenza A infection through suppression of group II innate lymphoid cells. Mucosal Immunol 2018; 11(1): 209-19.
48. Melo-Gonzalez F, Hepworth M R. Functional and phenotypic heterogeneity of group 3 innate lymphoid cells. Immunology 2017; 150(3): 265-75.
49. Shrestha S S, Swerdlow D L, Borse R H, et al. Estimating the burden of 2009 pandemic influenza A (H1N1) in the United States (April 2009-April 2010). Clin Infect Dis 2011; 52 Suppl 1: S75-82.
50. (WHO) WHO. Pandemic influenza vaccine manufacturing process and timeline. 2009.
51. Dawood F S, luliano A D, Reed C, et al. Estimated global mortality associated with the first 12 months of 2009 pandemic influenza A H1N1 virus circulation: a modelling study. Lancet Infect Dis 2012; 12(9): 687-95.
52. Vemula S V, Mittal S K. Production of adenovirus vectors and their use as a delivery system for influenza vaccines. Expert Opin Biol Ther 2010; 10(10): 1469-87.
53. Fineberg H V. Pandemic preparedness and response—lessons from the H1N1 influenza of 2009. N Engl J Med 2014; 370(14): 1335-42.
54. Zhang J, Tarbet E B, Feng T, Shi Z, Van Kampen K R, Tang D C. Adenovirus-vectored drug-vaccine duo as a rapid-response tool for conferring seamless protection against influenza. PloS one 2011; 6(7): e22605.
55. Pandey A, Singh N, Vemula S V, et al. Impact of preexisting adenovirus vector immunity on immunogenicity and protection conferred with an adenovirus-based H5N1 influenza vaccine. PloS one 2012; 7(3): e33428.
56. van Olphen A L, Mittal S K. Development and characterization of bovine x human hybrid cell lines that efficiently support the replication of both wild-type bovine and human adenoviruses and those with E1 deleted. Journal of Virology 2002; 76(12): 5882-92.
57. Sharma A, Bangari D S, Tandon M, Pandey A, HogenEsch H, Mittal S K. Comparative analysis of vector biodistribution, persistence and gene expression following intravenous delivery of bovine, porcine and human adenoviral vectors in a mouse model. Virology 2009; 386(1): 44-54.
58. Bangari D S, Mittal S K. Porcine adenoviral vectors evade preexisting humoral immunity to adenoviruses and efficiently infect both human and murine cells in culture. Virus research 2004; 105(2): 127-36.

What is claimed is:

1. A method for protection, prevention, or treatment against a viral infection by activating Innate Lymphoid Cells (ILCs) induced by a virus.

2. The method of claim 1, wherein the virus is non-replicating adenovirus or influenza virus.

3. The method of claim 2, wherein the non-replicating adenovirus is HAd-ΔE1E3 or HAd-H7HA virus

4. The method of claim 2, wherein the non-replicating adenovirus induces diverse and robust activation of ILCs in lungs lasting for a period of time.

5. The method of claim 4, wherein the activated ILCs are human ILCs selected from the group consisting of NK cells, ILC1, ILC2, and ILC3.

6. The method of claim 4, wherein the period of time is about 7 to 28 days.

7. The method of claim 4, wherein the non-replicating adenovirus induces infiltration of immune cells or expression of inflammatory cytokine and antiviral genes in lungs.

8. The method of claim 2, wherein the influenza virus is H1N1 or H3N2 influenza virus that induces activation of NK cells, ILC1, and ILC2 cells.

9. A method for protection against influenza epidemic or pandemic challenges where a strain-matched vaccine is not available, comprising administering a population with a non-replicating adenovirus to induce diverse and robust activation of ILCs.

10. The method of claim 9, wherein the non-replicating adenovirus is HAd-ΔE1E3 or HAd-H7HA virus.

11. The method of claim 9, wherein the ILCs are human ILCs selected from the group consisting of NK cells, ILC1, ILC2 and ILC3 that are activated in lungs for a period of time.

12. The method of claim 9, wherein protection against influenza epidemic or pandemic challenges is to contain the spread and reduce disease burden, limit disease severity, mitigate influenza-related deaths, reduce morbidity, or facilitate recovery.

13. A composition for protection, prevention, or treatment against a viral infection comprising an effective amount of a virus that is capable to induce diverse and robust activation of ILCs.

14. The composition of claim 13, wherein the virus is non-replicating adenovirus or influenza virus.

15. The composition of claim 14, wherein the non-replicating adenovirus is HAd-ΔE1E3 or HAd-H7HA virus that induces activation of ILCs selected from the group consisting of NK cells, ILC1, ILC2, and ILC3.

16. The composition of claim 17, wherein the influenza virus is H1N1 or H3N2 influenza virus that induces activation of NK cells, ILC1, and ILC2 cells.

17. The composition of claim 13, wherein the ILCs are activated in lungs and last for about 7-28 days.

18. The composition of claim 13, wherein the composition is administered via inhalation or injection.

19. A composition for protection against influenza epidemic or pandemic challenges where a strain-matched vaccine is not available, comprising an effective amount of a non-replicating adenovirus that is capable of inducing diverse and robust activation of ILCs.

20. The composition of claim 19, wherein the non-replicating adenovirus is HAd-ΔE1E3 or HAd-H7HA virus.
